SEQUENCE LISTING (10 sequences)

SEQ ID NO: 1 - DNA, 23 bp, Artificial Sequence
  Forward primer of ALP
accattcccacgtcttcacattt

SEQ ID NO: 2 - DNA, 22 bp, Artificial Sequence
  Reverse primer of ALP
agacattctctcgttcaccgcc

SEQ ID NO: 3 - DNA, 20 bp, Artificial Sequence
  Forward primer of RUNX-2
atttctcacctcctcagccc

SEQ ID NO: 4 - DNA, 20 bp, Artificial Sequence
  Reverse primer of RUNX-2
caacagccacaagttagcga

SEQ ID NO: 5 - DNA, 20 bp, Artificial Sequence
  Forward primer of BSP
cgaatacacgggcgtcaatg

SEQ ID NO: 6 - DNA, 24 bp, Artificial Sequence
  Reverse primer of BSP
gtagctgtactcatcttcataggc

SEQ ID NO: 7 - DNA, 20 bp, Artificial Sequence
  Forward primer of OCN
ggcgctacctgtatcaatgg

SEQ ID NO: 8 - DNA, 20 bp, Artificial Sequence
  Reverse primer of OCN
tcagccaactcgtcacagtc

SEQ ID NO: 9 - DNA, 19 bp, Artificial Sequence
  Forward primer of GAPDH
cctgttcgacagtcagccg

SEQ ID NO: 10 - DNA, 20 bp, Artificial Sequence
  Reverse primer of GAPDH
cgaccaaatccgttgactcc

(19) United States
(12) Patent Application Publication
(10) Pub. No.: US 2023/0285275 A1
(43) Pub. Date: Sep. 14, 2023

(54) BIOCOMPATIBLE STRUCTURE COMPRISING HOLLOW CAGE, AND MANUFACTURING METHOD THEREFOR

(71) Applicant: MEDIFAB CO., LTD., Seoul (KR)

(72) Inventors: Mi Sun CHA, Seoul (KR); Jae Hyup LEE, Seoul (KR)

(21) Appl. No.: 17/311,010

(22) PCT Filed: Dec. 5, 2019
(86) PCT No.: PCT/KR2019/017081

§ 371 Date: Jun. 4, 2021

(30) Foreign Application Priority Data
Dec. 7, 2018 (KR) 10-2018-0157468

(51) Int. Cl. A61K 9/00; A61P 19/00; A61K 38/18; A61K 38/30; A61K 38/40; A61K 31/663; A61K 9/06

(52) U.S. Cl. CPC A61K 9/0024; A61P 19/00; A61K 38/1875; A61K 38/1858; A61K 38/1841; A61K 38/1825; A61K 38/30; A61K 38/40; A61K 31/663; A61K 9/06

(57) ABSTRACT

The present disclosure relates to: a biocompatible structure comprising a hollow cage, the biocompatible structure comprising, in a surface thereof, one or more open chambers that are recessed inward and hold a biologically active substance; and a method of manufacturing the same. A biocompatible structure according to one aspect comprises a mixed solution of a hydrogel and a biologically active substance in a chamber so that an osteogenesis-promoting substance is released continuously over a long period of time while having initial release stability, and thus osteogenesis at bone defect sites may be improved.

### TECHNICAL FIELD

The present disclosure relates to a biocompatible structure including a hollow cage, and a method of manufacturing the same. This application is based on and claims priority to Korean Patent Application No. 10-2018-0157468, filed on Dec. 7, 2018, in the Korean Intellectual Property Office, the disclosure of which is incorporated by reference herein in its entirety.

### BACKGROUND ART

Repairing critical bone injuries is one of the most important tasks for orthopedic surgeons. Application of autografts and allografts is limited due to limited supply and the risks of complications of donors and disease transmission. Thus, in order to design a biocompatible bone replacement, tissue engineers have attempted to induce the growth of normal bone tissue by constructing a three-dimensional synthetic bone scaffold using various substances and attaching cells or growth factors thereto.

Bone morphogenetic protein-2 (BMP-2) belonging to the transforming growth factor-β super family is involved in processes of growth and development of bones and reconstruction of cartilage by promoting differentiation and maturation of osteoblasts. Therefore, because effective control of bioactivity and spatial-temporal presence is essential for effective bone regeneration, attempts have been made to develop biomaterial carriers maintained at a sufficient concentration in application regions to stimulate normal physiological mechanisms required for bone regeneration. Because the binding ability of BMP-2 to collagen sponge is high, approaches using a carrier, e.g., methods of adsorbing BMP-2 to collagen sponge or methods of soaking collagen sponge with BMP-2 are generally used. Research has been widely conducted on bioceramics including hydroxyapatite and tricalcium phosphate for transport of BMP-2. However, there are problems in that these lack the ability to be modified and it is difficult to modify continuous release of BMP-2.

Therefore, synthetic biodegradable polymers are advantageous in that mechanical properties, microstructures, and decomposition rates thereof may be considerably controlled by compositions and manufacturing techniques, and thus there is a need for extensive research into synthetic biodegradable polymers in bone tissue engineering applications.

### DESCRIPTION OF EMBODIMENTS

### Technical Problem

Provided is a biocompatible structure including a hollow cage including, in a surface thereof, one or more open chambers recessed inward and holding a biologically active substance.

Provided is a biocompatible structure including a hollow cage including, in a surface thereof, one or more open chambers recessed inward and holding a mixed solution of a hydrogel and a biologically active substance.

Provided is a method of manufacturing a biocompatible structure, the method including: preparing a hollow cage including one or more chambers in top and bottom surfaces; and loading a mixed solution of a hydrogel and a biologically active substance into the chambers.

### Solution to Problem

According to an aspect of the present disclosure, a biocompatible structure includes a hollow cage including, in a surface thereof, one or more open chambers recessed inward and holding a biologically active substance.

Specifically, the open chamber is used for releasing the loaded biologically active substance in vivo and one or more chambers may be formed on a straight line at upper and lower ends of the cage in the same size or different sizes. The chamber may be formed in a square shape with a length of one side in a range of 0.001 to 10 mm, 0.001 to 5 mm, 0.001 to 3.5 mm, 1 to 2.5 mm, ort to 1.5 mm.

In an embodiment, the open chamber is formed at the cage, and the open chamber may be formed in a groove shape open to the outside to communicate with the outside. The open chamber may be formed in the shape of a square groove, but the shape is not limited thereto, and the open chamber may be formed in various groove shapes open to the outside. The hollow cage, in which the open chambers are formed, may be formed in the shape of a cylinder, a square column, a triangular column, a pentagonal column, or a hexagonal column, or may have an irregular shape.

The open chamber may be formed on portions of the top and bottom surfaces of the cage and one or more open chambers may be formed on portions of the top and bottom surfaces of the cage. Specifically, referring to FIG. 2, when the cage is formed in a cylindrical shape, the open chamber may be formed on the top and bottom surfaces.

The open chambers may be formed on a portion of a side surface of the cage. Referring to FIG. 3, when the cage is formed in a square column shape, the open chamber may be formed on a side surface of the square column. However, positions of the open chambers are not limited to the top surface, the bottom surface, and the side surfaces, and the open chambers may be provided at various positions of the hollow cage.

According to an embodiment, the open chambers may be provided at the cage forming two or more layers. Referring to FIG. 3, a plurality of open chambers may be aligned in a straight line forming one layer, and then a plurality of open chambers may be aligned in a straight light forming another layer. The number of layers formed of the plurality of open chambers may be two, but is not limited thereto, and the open chambers may constitute a plurality of layers.

As a method of loading the biologically active substance into the open chamber, a method of directly loading the substance using a syringe or a stacking method using 3D bioprinting may be used. In an embodiment, when one or more chambers having the same size are formed, not only a strength of the biocompatible structure may be uniformly maintained over the entire area, but also the release of the biologically active substance may be controlled uniformly over the entire area of the biocompatible structure. In another embodiment, when one or more chambers with different sizes are formed, the chambers may constitute two or more layers. The chambers may form 2 or more, 3 or more, 4 or more, 5 or more, 6 or more, or 7 or more, or 8 or more layers. When the chambers form two or more layers, strength of each portion of the biocompatible structure may be adjusted, and a release rate of the biologically active substance may also be adjusted in accordance with the size of the chamber.

The hollow cage may have a cylindrical shape, a square column shape, a triangular column shape, a pentagonal column shape, a hexagonal column shape, or an irregular shape. The shape of the hollow cage is not limited to those described above and may include all shapes realized by 3D printing including a shape of an area to which the cage is implanted. In an embodiment, the cage having a cylindrical shape may have a diameter×height value of 0.001 to 15×0.001 to 15 mm, 10 to 13×1 to 4 mm, 8 to 13×1.5 to 3.5 mm, 8 to 10×1.5 to 2.5 mm, or 6 to 8×1.5 to 2.5 mm. In this case, when the size of the cage having the cylindrical shape is less than the above ranges, the biologically active substance may not be effectively delivered to a target region. When the size of the cage is greater than the above ranges, it is difficult to insert the cage into a living body.

According to an aspect of the present disclosure, a biocompatible structure includes a hollow cage including, in a surface thereof, one or more open chambers recessed inward and holding a mixed solution of a hydrogel and a biologically active substance.

As used herein, the term “biocompatible structure” refers to a structure that is not substantially toxic to the human body, is chemically inactive, and has no immunogenicity, and the structure may be manufactured as a three-dimensional artificial organ, a scaffold (i.e., bio support), a drug delivery, or the like which are precisely structured using a bioprinter and applied to a living body.

The hollow cage may be made of a polymeric material. Specifically, the polymeric material may be a biocompatible or biodegradable material.

As used herein, the term “biocompatible material” refers to a substance that is not substantially toxic to the human body, is chemically inactive, and has no immunogenicity. The term “biodegradable material” refers to a substance degradable by a body fluid or a microorganism in a living body.

In this case, the biocompatible or biodegradable material may be hyaluronic acid, polyester, polyhydroxyalkanoate (PHA), poly(lactic acid), poly(α-hydroxy acid), poly(β-hydroxy acid), poly(3-hydroxybutyrate-co-valerate) (PHBV), poly(3-hydroxypropionate) (PHP), poly(3-hydroxyhexanoate) (PHH), poly(4-hydroxy acid), poly(4-hydroxybutyrate), poly(4-hydroxyvalerate), poly(4-hydroxyhexanoate), poly(esteramide), polycaprolactone, polylactide, polyglycolide, poly(lactide-co-glycolide) (PLGA), polydioxanone, polyorthoester, polyether ester, polyanhydride, poly(glycolic acid-co-trimethylene carbonate), polyphosphoester, polyphosphoester urethane, poly(amino acid), polycyanoacrylate, poly(trimethylene carbonate), poly(iminocarbonate), poly(tyrosine carbonate), polycarbonate, poly(tyrosine arylate), polyalkylene oxalate, polyphosphazenes, PHA-PEG, an ethylene vinyl alcohol copolymer (EVOH), polyurethane, silicone, polyester, polyolefin, a polyisobutylene-ethylene-α-olefin copolymer, a styrene-isobutylene-styrene triblock copolymer, an acrylic polymer or copolymer, a vinyl halide polymer or copolymer, polyvinyl chloride, polyvinyl ether, polyvinyl methyl ether, polyvinylidene halide, polyvinylidene fluoride, polyvinylidene chloride, polyfluoroalkene, polyperfluoroalkene, polyacrylonitrile, polyvinyl ketone, polyvinyl aromatic, polystyrene, polyvinyl ester, polyvinyl acetate, an ethylene-methyl methacrylate copolymer, an acrylonitrile-styrene copolymer, ABS resin, an ethylene-vinyl acetate copolymer, polyamide, alkyd resin, polyoxymethylene, polyimide, polyether, polyacrylate, polymethacrylate, polyacrylic acid-co-maleic acid, chitosan, dextran, cellulose, heparin, alginate, inulin, starch or glycogen, and may be, hyaluronic acid, polyester, polyhydroxyalkanoate (PHA), poly(α-hydroxy acid), poly(β-hydroxy acid), poly(3-hydroxybutyrate-co-valerate) (PH BV), poly(3-hydroxyhydroxypropionate) (PHP), poly(3-hydroxyhexanoate; PHH), poly(4-hydroxy acid), poly(4-hydroxybutyrate), poly(4-hydroxyvalerate), poly(4-hydroxyhexanoate), poly(esteramide), polycaprolactone, polylactide, polyglycolide, poly(lactide-co-glycolide) (PLGA), polydioxanone, polyorthoester, polyetherester, polyanhydride, poly(glycolic acid-co-trimethylene carbonate), polyphosphoester, polyphosphoesterurethane, poly(amino acid), polycyanoacrylate, poly(trimethylene carbonate), poly(iminocarbonate), (tyrosine carbonate), polycarbonate, poly(tyrosine arylate), polyalkylene oxalate, polyphosphazenes, PHA-PEG, chitosan, dextran, cellulose, heparin, alginate, inulin, starch, or glycogen.

The hydrogel may be a mixture of alginate and gelatin. Specifically, the biologically active substance needs to be loaded in the chamber to be efficiently delivered to a target region. Therefore, the hydrogel inhibits explosive release of the biologically active substance in the early stage and plays a role in releasing the biologically active substance over a long period of time.

In addition, the biologically active substance may be an osteogenesis-promoting substance. Specifically, the osteogenesis-promoting substance may be a bone morphogenetic protein (BMP), a platelet-derived growth factor (PDGF), a transforming growth factor beta (TGF-beta), a basic fibroblast growth factor (bFGF), an insulin-like growth factor 1 (IGF-1), lactoferrin, and bisphosphonate. In this regard, the Bisphosphonate may be etidronate, clodronate, tiludronate, pamindronate, alendronate, risendronate, ibandronate, or zolendronate.

In addition, the hydrogel solution is used for adjusting an initial release rate of the osteogenesis-promoting substance, and the hydrogel may be mixed with the biologically active substance in a weight ratio (w/w) of 0.5 to 9.5:9.5 to 0.5, 0.5 to 8:8 to 0.5, 0.5 to 5:5 to 0.5, 0.5 to 3.5:3.5 to 0.5, or 1 to 1.5:1.5 to 1. In this regard, when the mixing ratio of the hydrogel to the biologically active substance is less than the ranges described above, the initial release rate may decrease, and thus there is a problem in that the biologically active substance cannot be efficiently delivered to the target region. When the mixing ratio is greater than the ranges described above, the release of the biologically active substance explosively increases in the early stage, and thus there is a problem in that the biologically active substance cannot be continuously delivered to the target region.

In an embodiment, cells or tissue may further be loaded into the open chambers. The cells may include, for example, cells to be transplanted into defect sites, cells to be differentiated into tissue or other cells, or cells to be used in tissue regeneration. The cells may be, for example, stem cells, sensory cells, brain cells, germ cells, epithelial cells, immune cells, chondrocytes, osteocytes, cancer cells, or any combination thereof. The stem cells may refer to cells having differentiation potency, and the cells having differentiation potency may be, for example, blast cells, hepatocytes, fibroblasts, myoblasts, adult stem cells, mesenchymal stem cells, adipose-derived mesenchymal stem cells, bone marrow-derived mesenchymal stem cells, nerve-derived mesenchymal stem cells, placenta-derived mesenchymal stem cells, umbilical cord blood stem cells, or any combination thereof. As described above, when the mixed solution of the hydrogel and the osteogenesis-promoting substance is loaded into the chamber of the biocompatible structure according to an embodiment, the osteogenesis-promoting substance is gradually accumulated at the target region due to the initial release stability of the osteogenesis-promoting substance, and thus bones regularly grow and bone density increases.

Another aspect of the present disclosure provides: a composition for restoring biological tissue including the biocompatible structure; a use of the biocompatible structure for restoring biological tissue; and a method of restoring biological tissue including implanting the biocompatible structure into an individual.

As used herein, the term “biological tissue” refers to tissue, particularly, tissue to which tissue alternative, particularly, the biocompatible structure, is implanted for tissue restoration, tissue well known in the field of tissue engineering, for example, osseous tissue, without being limited thereto.

As used herein, the term “individual” refers to an object requiring tissue repair, and more particularly, mammals such as humans or non-human primates, mice, dogs, cats, horses, and cows.

According to another aspect of the present disclosure, a method of manufacturing a biocompatible structure includes: preparing a hollow cage including one or more chambers on at least one surface of the top and bottom surfaces; and loading a mixed solution of a hydrogel and a biologically active substance into the chambers.

The manufacturing method of an embodiment includes preparing a hollow cage including one or more chambers on the top and bottom surfaces. Detailed descriptions of the hollow cage are as described above. The hollow cage may be prepared by a fused deposition modeling (FDM) method to deposit a material through a melting head. In this regard, the material may be a biocompatible or a biodegradable polymeric material.

The manufacturing method of an embodiment includes loading the mixed solution of the hydrogel and the biologically active substance into the chambers. Detailed descriptions of the hydrogel and the biologically active substance are as described above. The loading of the mixed solution may be performed by any method well known in the art.

In addition, the mixed solution of the hydrogel and the biologically active substance may be prepared by warming up the hydrogel and adding the biologically active substance thereto, and mixing. Specifically, the warming up may be performed at a temperature of 4° C. to 75° C., 25° C. to 60° C., 35° C. to 45° C., or 35° C. to 40° C. In this case, when the temperature for warming up is lower than the above ranges, the hydrogel is not sufficiently melted causing a problem in that it is difficult to mix the hydrogen with the biocompatible material. When the temperature is higher than the above ranges, the hydrogel is solvated causing a problem in that it is difficult to load the mixed solution with the biologically active substance into the chambers.

### Advantageous Effects of Disclosure

The biocompatible structure according to an embodiment includes the hollow cage including, in a surface thereof, one or more open chambers recessed inward and holding a biologically active substance. Because a hydrogel and an osteogenesis-promoting substance are loaded into the chambers, osteogenesis may occur in bone defect sites. In addition, the hollow cage may effectively deliver a drug and enable regular osteogenesis at bone defect sites in the treatment of bone defects by inhibiting explosive release of the osteogenesis-promoting substance in the early stage and enabling sustained release over a long period of time.

### BRIEF DESCRIPTION OF DRAWINGS

FIGS. 1A and 1B show cage scaffolds according to an embodiment. FIG. 1A shows a disk-shaped cage having 6 holes on the front and back surfaces respectively imaged using CAD software of SolidWorks, and FIG. 1B shows a 3D-printed cage filled with about 35 μL of a biogel.

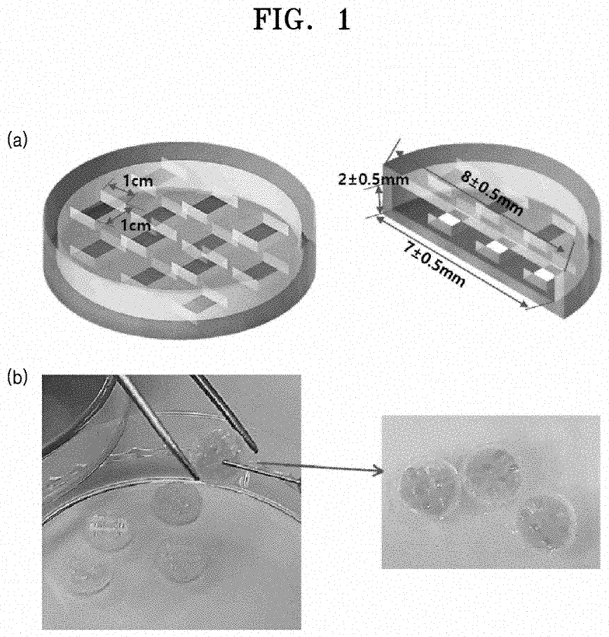

FIG. 2 is a cross-sectional view of a hollow cage having a cylindrical shape according to an embodiment.

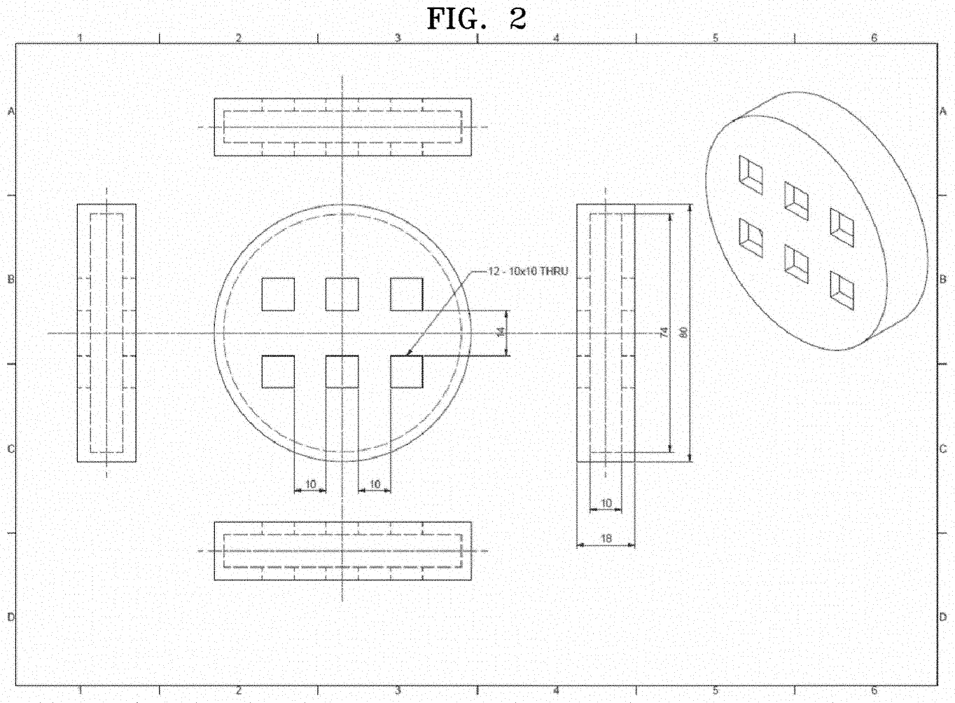

FIG. 3 is a cross-sectional view of a hollow cage having a square column shape according to an embodiment.

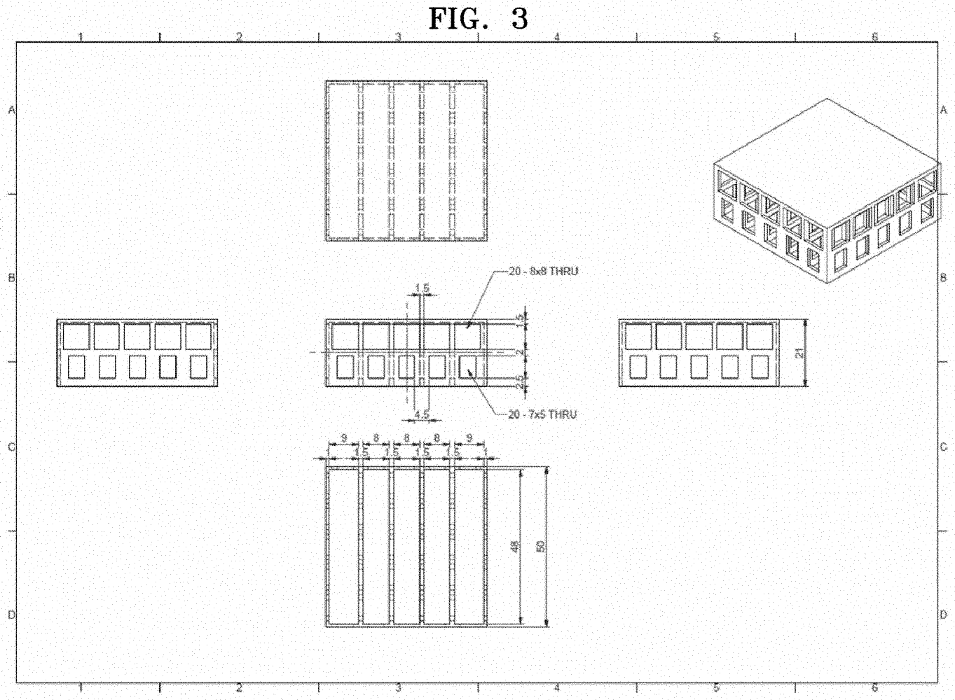

FIG. 4 is a graph illustrating an in vitro release pattern of BMP-2.

FIG. 5 is a graph illustrating biocompatibility of a scaffold and a biogel according to an embodiment.

FIG. 6A is a graph illustrating in vitro ALP activity of BMP-2 released from a cage including a biogel.

FIG. 6B shows ALP staining results of BMP-2 released from a cage including a biogel.

FIG. 7A is a graph for evaluating the effects of a BMP-2 release profile on osteogenic differentiation-related gene ALP in vitro.

FIG. 7B is a graph for evaluating the effects of a BMP-2 release profile on osteogenic differentiation-related gene Runx-2 in vitro.

FIG. 7C is a graph for evaluating the effects of a BMP-2 release profile on osteogenic differentiation-related gene BSP in vitro.

FIG. 7D is a graph for evaluating the effects of a BMP-2 release profile on osteogenic differentiation-related gene OCN in vitro.

FIG. 8 shows reconstructed micro-CT images of osteogenesis in a cage system.

FIG. 9 show photographs showing histological images of a rat calvarial defect model having a defect with a critical size.

FIG. 10 shows reconstructed micro-CT images of osteogenesis in a cage system.

FIG. 11 shows photographs showing histological images of a rat ectopic model.

FIG. 12 is a schematic diagram illustrating a process of preparing a scaffold according to an embodiment and implanting the scaffold into a defect site of a rabbit tibia.

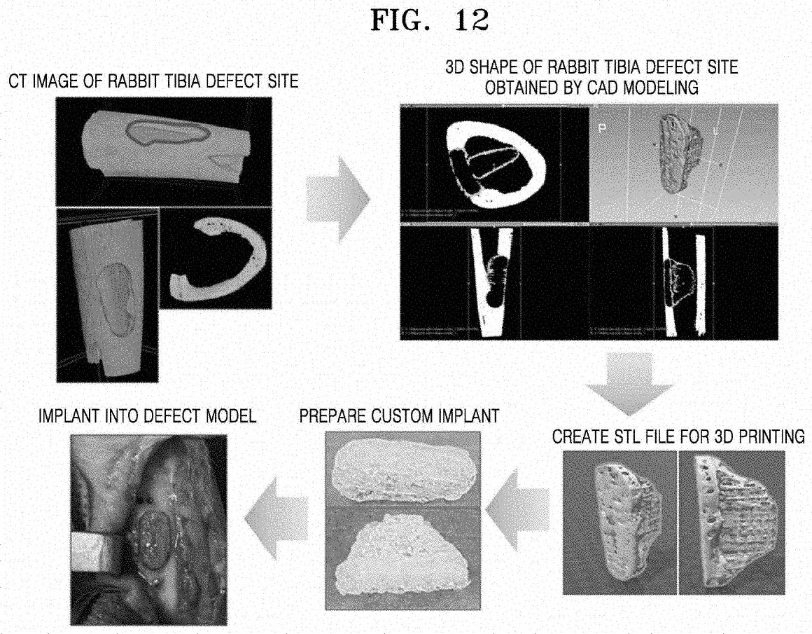

FIG. 13A shows photographs for identification of the degree of osteogenesis of a defect site after implanting a scaffold of Example 1 into a rabbit tibia.

FIG. 13B shows photographs for identification of the degree of osteogenesis of a defect site after implanting a scaffold of Example 2 into a rabbit tibia.

FIG. 13C shows photographs for identification of the degree of osteogenesis of a defect site after implanting a scaffold of Example 4 into a rabbit tibia.

FIG. 13D shows photographs for identification of the degree of osteogenesis of a defect site after implanting a scaffold of Comparative Example 5 into a rabbit tibia.

### MODE OF DISCLOSURE

Hereinafter, the present disclosure will be described in more detail with reference to the following examples. However, the following examples are merely presented to exemplify the present disclosure, and the scope of the present disclosure is not limited thereto.

### Preparation Example

Pure PLA filaments were purchased from MakerBot (New York City, N.Y., USA) for FDM 3D printing. A biogel based on alginate and gelatin was supplied from MediFab Co. Ltd. (Seoul, Korea). All other compounds were purchased from Sigma-Aldrich (St. Louis, Mo., USA) and were used as obtained unless otherwise stated.

### EXAMPLES

### Example 1. Designing and Preparing of Scaffold

A hollow cage with a rectangular hole was designed using solid modeling software (SolidWorks®, Dassault Systemes SolidWorks Corp.) and the design was stored as a stereolithography file (.stl). Then, the file was converted into 3D printing codes using 3D printer software and input to a 3D printer. In the printer, a cartridge was installed to supply a PLA filament to the 3D printer (Replacator™2, Makerbot). Also, the melted PLA filament was extruded through a heated metal nozzle (diameter: 0.2 mm, moving horizontally and vertically) at 205° C. and placed in a container to prepare a scaffold.

### Example 2. Preparation of Scaffold Loaded with Mixed Solution of Biogel and BMP-2

A biogel was warmed up at 37° C. for 30 minutes in accordance with manufacturer's recommendations. After uniformly mixing BMP-2 and the warmed-up biogel at 250 ng/ml each, the mixed solution of the biogel and BMP-2 was loaded into the cage via the rectangular hole of the scaffold prepared in Example 1. The cage loaded with the mixed solution of the biogel and BMP-2 was immersed in a casting buffer (Medifab Co. Ltd., Seoul, Korea) at 4° C. for 20 minutes for gelation. Thereafter, the cage was washed with a PBS buffer to prepare a scaffold.

### Example 3. Preparation (2) of Scaffold Loaded with Mixed Solution of Biogel and BMP-2

The scaffold prepared in Example 1 was sterilized under UV light with hands washed three times with DPBS. Then, the mixed solution of the biogel and BMP-2 prepared in the same manner as in Example 2 was loaded into the cage to prepare a scaffold.

### Example 4. Preparation of Scaffold Loaded with Biogel, BMP-2 and Mesenchymal Stem Cell

The scaffold prepared in Example 1 was sterilized under UV light with hands washed three times with DPBS. Then, a mixed solution of the biogel, BMP-2 and mesenchymal stem cells prepared in the same manner as in Example 2 was loaded into the cage to prepare a scaffold.

### COMPARATIVE EXAMPLE

### Comparative Example 1. Scaffold Loaded with Biogel

A scaffold was prepared in the same manner as in Example 2, except that the biogel was loaded into the scaffold prepared in Example 1 at 250 ng/ml.

### Comparative Example 2. Scaffold Loaded with BMP-2

A scaffold was prepared in the same manner as in Example 2, except that 40 μL of BMP-2 was loaded into the scaffold prepared in Example 1.

### Comparative Example 3. Scaffold Loaded with Biogel (2)

A scaffold was prepared in the same manner as in Example 3, except that the biogel was loaded into the scaffold prepared in Example 1 at 250 ng/ml.

### Comparative Example 4. Scaffold Loaded with BMP-2 (2)

A scaffold was prepared in the same manner as in Example 3, except that 40 μL of BMP-2 was loaded into the scaffold prepared in Example 1.

### Comparative Example 5. Scaffold Loaded with Mesenchymal Stem Cells

A scaffold was prepared in the same manner as in Example 4, except that mesenchymal stem cells (MSCs) were loaded into the scaffold prepared in Example 1 at 1×10⁶ cell/ml.

### Experimental Example

In Vitro Analysis

(1) Measurement of Released Amount of BMP-2

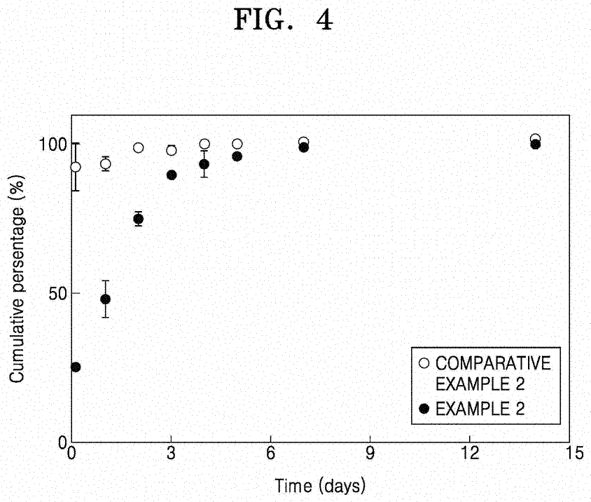

The scaffolds according to Example 2 and Comparative Example 2 were placed in an upper compartment of a Trans-well system (SPL Lifesciences, Korea). A release medium (HBSS) in a lower compartment was refreshed at several time points. The release was determined by measuring amounts of BMP-2 (2.5 μg/ml) in a release medium by ELISA. Data was expressed as cumulative amount of a total input. Results of measuring released amounts of BMP-2 in vitro are shown in FIG. 4.

As shown in FIG. 4, in the case of Comparative Example 2, it was confirmed that BMP-2 was rapidly released so that 90% of BMP-2 was released after 3 hours and the concentration of BMP-2 reached below a detectable limit after 2 days. On the contrary, in the case of Example 2, a unique profile in which BMP-2 was explosively released for initial 4 days, followed by a secondary sustained release was observed. Specifically, about 85% of BMP-2 was gradually released in Phase 1 and then the release rate gradually decreased thereafter. That is, the scaffold loaded with the mixed solution of BMP-2 and the biogel may effectively deliver a drug in treatment of bone defects by inhibiting an explosive release of the drug in the early stage and continuously releasing the drug over a long period of time.

(2) Measurement of Biocompatibility

Biocompatibility and toxicity of the scaffolds of Examples 1 and 2 and Comparative Example 1 were measured with a Cellrix® viability measurement kit (Medifab Co. Ltd., Korea). Implants prepared in Examples 1 and 2 and Comparative Example 1 were aliquoted into a 24-well plate containing mMSCs (1×10³ cells) and the cells were cultured in 0.5 ml of a MEM a medium (Gibco, Life Technologies). The next day after fixation, a cell culture insert with an implant pore size of 8 μm according to Examples 1 and 2 and Comparative Example 1 was added to the medium in which the cells were aliquoted. At every measurement time, the insert and the case were removed, and the kit was added and mixed. After 30 minutes of culturing, absorbance was measured twice at a wavelength of 450 nm. BMP-2 was used as a positive control.

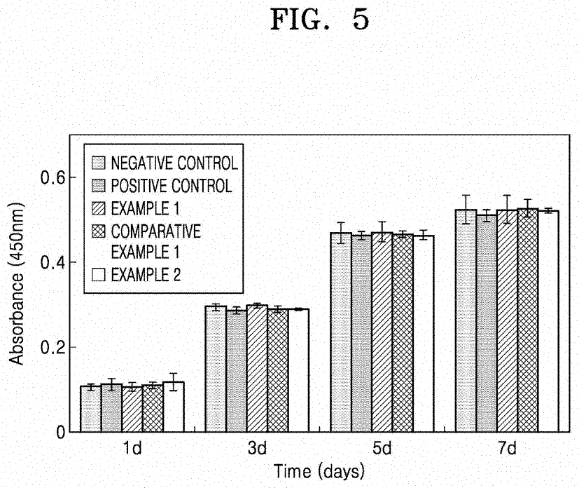

As a result, as shown in FIG. 5, it was confirmed that the number of cells of the control gradually increased over time and cell proliferation patterns of Examples 1 and 2 and Comparative Examples 2 and 3 were similar thereto. That is, the scaffolds prepared in Examples 1 and 2 and Comparative Examples 2 and 3 had excellent biocompatibility without cytotoxicity.

(3) ALP Assay and Staining

ALP activity of mMSCs was analyzed to determine biological activity of BMP-2 released from the scaffolds according to Examples 1 and 2 and Comparative Examples 1 and 2. Specifically, the activity of alkaline phosphatase (ALP), as a marker of the early stage of osteogenic differentiation was analyzed based on conversion of colorless p-nitrophenylphosphate into colored p-nitrophenol. The cells were washed twice with DPBS and lysed with a 0.2% Triton X-100 solution. ALP activity of cell lysates was analyzed using p-nitrophenylphosphate as a substrate. After culturing the cells at room temperature for 30 minutes, the biological activity was determined using a released amount of p-nitrophenol. The color change was measured using a spectrometer at 405 nm. BMP-2 was used as a positive control.

For ALP staining, the cells were washed with DPBS and fast blue PR salt was added to each well. Then, the cells were cultured at room temperature for 10 minutes and observed with an optical microscope.

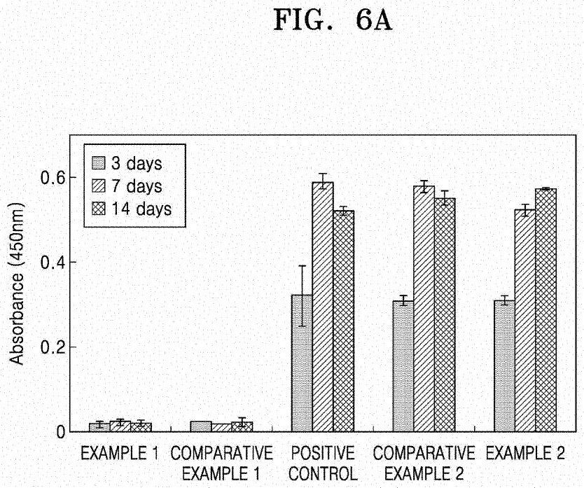

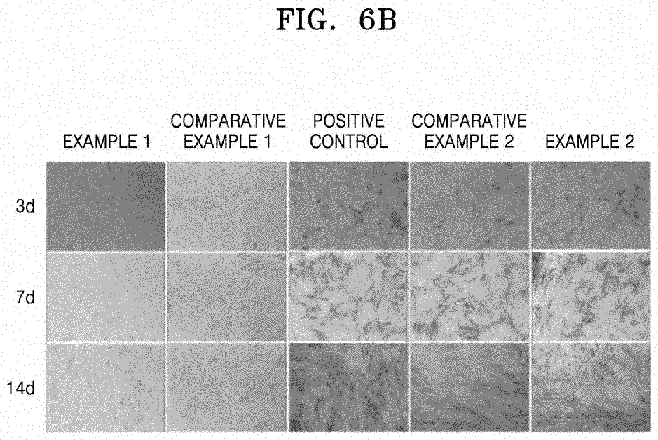

As a result, as shown in FIG. 6, the ALP protein activity of the mMSCs did not increase in Example 1 and Comparative Example 1, and thus it was confirmed that effects of the scaffold and biogel were not significant in osteogenic differentiation. However, in the cases of Example 2 and Comparative Example 2, it was confirmed that the ALP protein activity increased over the entire culture period. Also, while the ALP activity significantly increased in the early stage (7 days) by BMP-2 and according to Comparative Example 2, it was confirmed that the ALP activity continuously increased up to 14 days of culturing in the case of Example 2. That is, BMP-2 released from the scaffold according to Example 2 exhibited biological activity for at least 2 weeks.

(4) Analysis of Osteogenic Differentiation

Effects of BMP-2 released from the scaffolds according to Examples 1 and 2 and Comparative Examples 1 and 2 on osteogenic differentiation were analyzed. Specifically, gene expression was evaluated by real-time PCR using particular primer sets, and the primer sets used herein are shown in Table 1 below. A total RNA was isolated from in vitro cultures on day 3, day 7, and day 14 using a RNeasy mini kit (Quiagen, USA), and subjected to reverse transcription using a high capacity cDNA reverse transcription kit (Intron Biotechnology, Korea). After synthesizing cDNA, real-time PCR (LightCycler® instrument, Roche) was performed in ALP, Runx-2, OCN, and BSP. GAPDH was used as an endogenous control.

TABLE 1

| Gene | Forward primer | Reverse primer |
|---|---|---|
| ALP | ACC ATT CCC ACG TCT | AGACATTCTCTCGTTCACCGCC |
| | TCA CAT TT | |
| RUNX-2 | ATT TCT CAC CTC CTC | CAA CAG CCA CAA GTT AGC |
| | AGC CC | GA |
| BSP | CGA ATA CAC GGG CGT | GTA GCT GTA CTC ATC TTC |
| | CAA TG | ATA GGC |
| OCN | GGC GCT ACC TGT ATC | TCAGCCAACTCGTCACAGTC |
| | AAT GG | |
| GAPDH | CCT GTT CGA CAG TCA | CGACCAAATCCGTTGACTCC |
| | GCC G | |

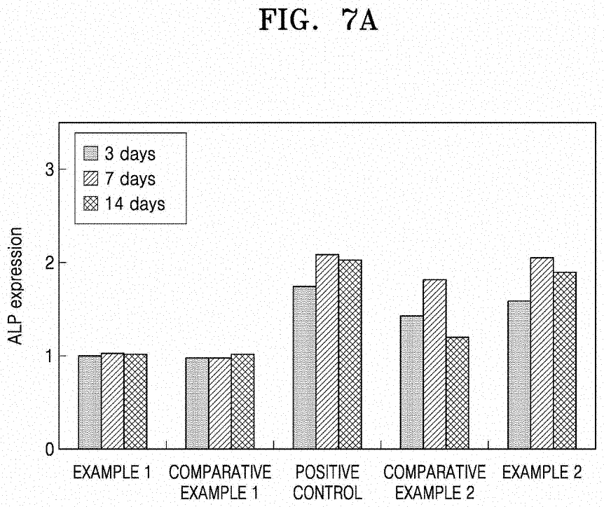

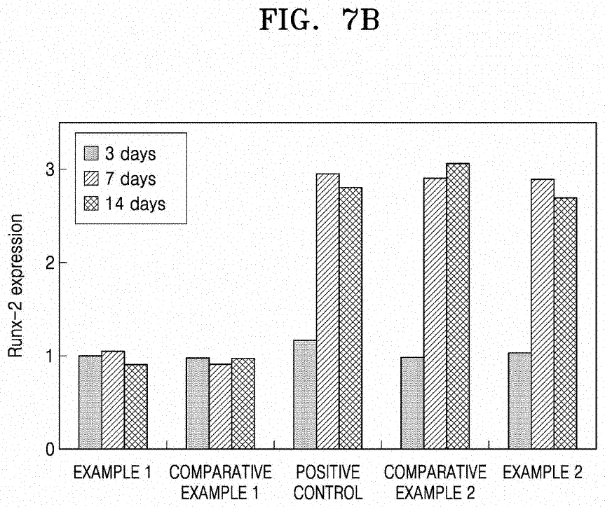

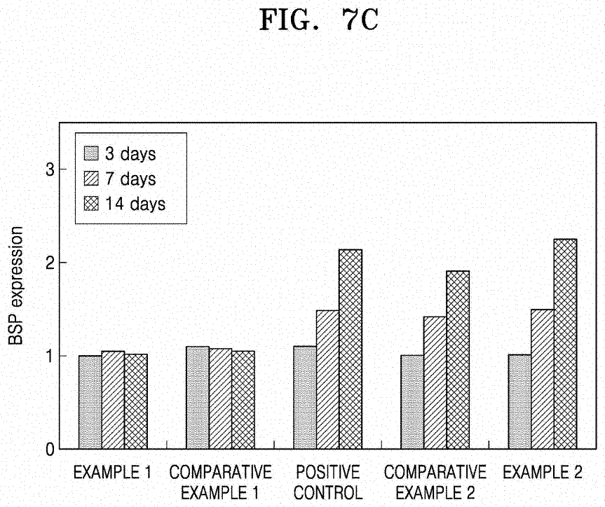

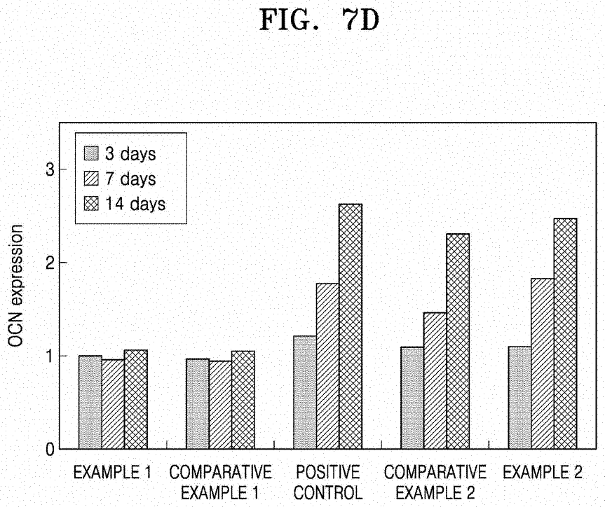

As a result, as shown in FIG. 7, it was confirmed that expression levels of the osteogenic marker gene significantly increased in both Example 2 and Comparative Example 2. Also, while the gene expression was significantly increased in the early stage (7 days) by BMP-2 and according to Comparative Example 2, it was confirmed that the gene expression was continuously increased up to 14 days of culturing in the case of Example 2. That is, BMP-2 released from the scaffold according to Example 2 may exhibit osteogenic differentiation activity for at least 2 weeks.

In Vivo Analysis

(1) Rat Calvarial Defect

A procedure related to use of a rat calvarial defect animal model was approved by the international animal care and use committee (SSBMC IACUC No. 2016-0044). 41 male Sprague-Dawley (SD) rats aged 8 weeks (200 to 220 g) were used for the animal test. All animals were allowed to freely access to feeds and drinking water and stored freely without a particular pathogen. The experiment was conducted after a one-week stabilization period. A surgical procedure was performed under semi-sterile conditions after anesthetizing the rats by intraperitoneal injection of Zoletil (20 mg/kg) mixed with Xylazine (10 mg/kg). A surgical site was shaved and sterilized with a povidone iodine solution. The scalp was vertically incised and periosteum was dissected. An 8 mm calvarial defect was created using a commercially available trephine burr. The defect site was irrigated with normal saline to minimize a speed of the trephine burr and minimize thermal damage. Also, the defect site was carefully preserved to prevent sagittal sinus bleeding and impairment of bone healing without damaging dura mater. After the defect was created, each of the scaffolds prepared in Examples 1 and 2 and Comparative Examples 1 and 2 was inserted thereinto. Then, the scalp was sutured. 100 mg/kg cefazolin was injected into the rats and stored under temperature and humidity conditions in which a dark:light cycle was adjusted 12:12. All animals were sacrificed after deep anesthesia in a CO2 chamber for 8 weeks after implanting the scaffold. Then, the implant inserted into the animal was carefully harvested with peripheral parietal, interparietal, and frontal. All samples were immobilized with 10% formalin for micro-CT evaluation and histological assessment.

1-1) Microcomputed Tomography (CT) Imaging and Analysis

Tomographic images were obtained using a Skyscan 1172 micro-CT scanner (Bruker, Belgium). Specifically, the samples harvested in (1) above were scanned with a pixel of 9.85 μm, an Al filter of 0.5 mm, an energy of 59 kV, and a current of 169 μA at a rotation step of 0.4°. Then, the tomographic images were reconstructed using a NRecon package (Bruker, Belgium) and analyzed using a CT Analyzer software (CT-An, Bruker, Belgium). A threshold value of newly formed bone was set based on a native bone. Trabecular bone thickness (Tb.Th), bone volume (BV), and percent bone volume (BV/TV) of regions of interest (ROIs) formed at the defect site and having a diameter of 8 mm were calculated, and the results are shown in Table 2 below:
BS. BV: Bone surface/Bone volume
Tb.Sp: Trabecular Separation
Tb.N: Trabecular Number
Tb.Pf: Trabecular bone pattern factor
SMI: Structure model index
DA: Degree of Anisotropy

TABLE 2

| | | Bone | | Trabec- | | Trabec- | | Degree |
|---|---|---|---|---|---|---|---|---|
| | ercent | surface/ | Trabec- | ular | Trabec- | ular bone | Structure | of |
| | bone | bone | ular bone | sepa- | ular bone | pattern | model | aniso- |
| | volume | volume | thickness | ration | number | factor | index | tropy |
| Group | BV.TV) | (BS.BV) | (Tb.Th) | (Tb.Sp) | (Tb.N) | (Tb.Pf) | (SMI) | (DA) |
| (no.) | Average (SD) | | | | | | | |
| Example 1 | 3.224 | 20.145 | 0.195 | 1.74 | 0.166 | 5.023 | 2.026 | 2 |
| (9) | (2.054) | (2.36) | (0.022) | (0.313) | (0.129) | (9.724) | (20.21) | (0.473) |
| Example | 23.414 | 16.73 | 0.164 | 0.726 | 1.434 | −17.572 | −3.962 | 1.718 |
| 2(9) | (4.518) | (1.799) | (0.012) | (0.168)^{ab} | (0.304)^{ab} | (4.28)^{ac} | (1.533)^{abc} | (0.137)^{b} |
| Comparative | 2.945 | 19.631 | 0.178 | 2.025 | 0.169 | −6.693 | −0.424 | 2.275 |
| Example | (1.276) | (4.044) | (0.034) | (0.282) | (0.077) | (8.014) | (1.697) | (0.482) |
| 1 (11) | | | | | | | | |
| Comparative | 20.886 | 17.049 | 0.194 | 0.701 | 1.104 | 5.34 | 1.935 | 1.802 |
| Example | (7.67)^{ab} | (2.918) | (0.031) | (0.366)^{ab} | (0.437)^{ab} | (15.973) | (3.825) | (0.207)^{b} |
| 2 (12) | | | | | | | | |

^{a}Comparison with Example 1, p < 0.05
^{b}Comparison with Comparative Example 1, p < 0.05
^{c}Comparison with Comparative Example 2, p < 0.05

As a result, as shown in Table 2, in the case of Example 2 and Comparative Example 2, it was confirmed that the percent bone volumes were higher by about 10 times, Tb.N and Tb.Th were higher, and Tb.Sp was lower than those of Example 1 and Comparative Example 1. Also, in the case of Example 2 including the biogel, it was confirmed that the percent bone volume was higher and the Tb.Pf and SMI were significantly lower than those of Comparative Example 2. That is, the difference in the parameters as described above indicates that addition of the biogel including BMP-2 for sustained release may increase formation of new bones having considerably high continuity and enclosed cavities.

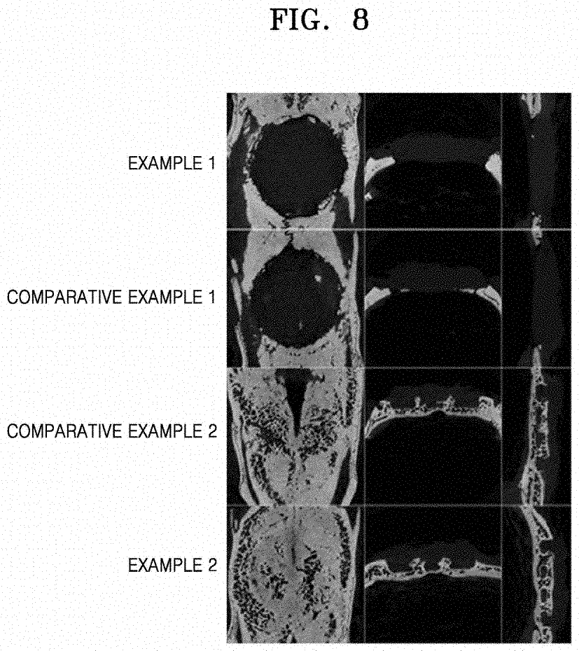

Also, as shown in FIG. 8, in Example 1 and Comparative Example 1, very little formation of bones was confirmed in the bone defect sites after 8 weeks from the surgical procedure. On the contrary, it was confirmed that the growth of bone was induced in a large volume in Example 2 and Comparative Example 2. Also, mineralized tissue was well dispersed and formed and the undecalcified cross-section of calvarial bone was re-confirmed by results of microcomputed-CT.

1-2) Histological Assessment

The samples harvested in (1) above were immobilized with 10% formalin and sequentially dehydrated with 80% to 100% ethyl alcohol and immersed and embedded in a Technovit 7200 resin (EXAKT, Germany). Then, the resin was cured by a polymerization system (EXAKT, Germany). The cured resin block was sliced into sections having a thickness of 200 μm using a cutting system (EXAKT, Germany) and ground to a thickness of 50 μm using a grinding system (EXAKT, Germany). Then, the ground pieces were stained with Hematoxylin & Eosin and observed. Formation of bones in the scaffold was observed using a microscope or a digital camera.

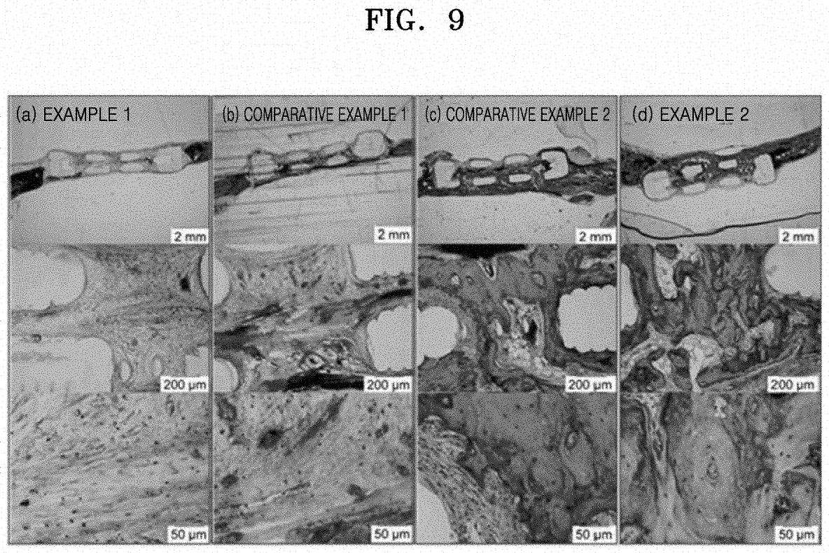

As a result, as shown in FIG. 9, it was confirmed that the growth of new bones was induced in a large volume in Example 2 and Comparative Example 2.

(2) Rat Ectopic Ossification Model

A procedure related to use of a rat ectopic ossification animal model was approved by the international animal care and use committee (SSBMC IACUC No. 2016-0044). 12 SD rats aged 12 weeks (375 to 400 g) were used for the animal test. Monitoring, stabilizing, and feeding of the animals were performed in the same manner as in (1) described above. The rats were anesthetized and shaved, and the exposed skin was sterilized with a povidone iodine solution in the same manner as in (1) above. The skin was incised to a length of about 7 cm, and iliocostal muscles were exposed using fingers and a gauze. Four pouches each having a length of 10 mm and a depth of 10 mm were made in muscles of both sides using a scalpel and a hemostats. The pouches on the same side were separated from each other by about 15 mm to prevent contact. After implanting the scaffolds prepared in Examples 1 and 2 and Comparative Examples 1 and 2, the muscle layer was sutured using a Vicryl and the skin was sutured with nylon. All animals were scarified after 6 weeks from the surgical procedure in the same manner as in (1) above. Iliocostal muscles of both sides were harvested and immobilized with 10% formalin for micro-CT evaluation and histological assessment.

2-1) Microcomputed Tomography (CT) Imaging and Analysis

The samples harvested in (2) above were scanned using the same device as that of Example 8-1 above with a pixel of 9.85 μm, an energy of 49 kV, and a current of 200 μA at a rotation step of 0.7 μm. Then, the microcomputed CT image was treated with the same method shown in 1-1) above. Because the scaffold is translucent at radio frequencies, only the volume of new bones was measured, and the results are shown in Table 3 below.

TABLE 3

| | Comparative Example 2 | Example 2 |
|---|---|---|
| Parameter | (n = 12) | (n = 12) |
| Bone volume | 2.663 ± 3.116 | 5.926 ± 2.539* |

As a result, as shown in Table 3, in the case of Example 2 including the biogel, it was confirmed that the volume of newly formed bones significantly increased compared to that of Comparative Example 2 by 2.2 times

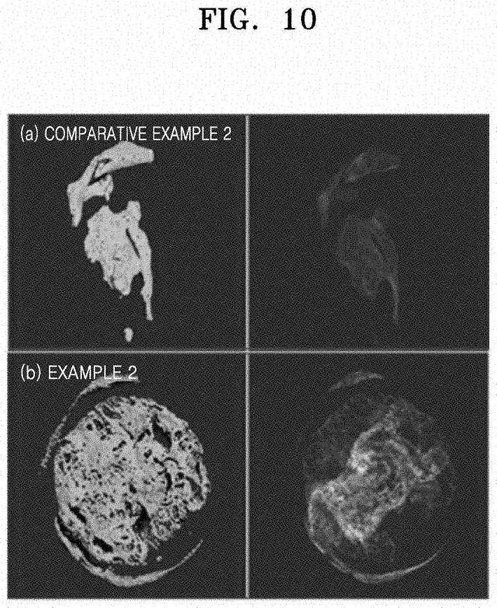

Also, as shown in FIG. 10, it was confirmed that the newly formed bones of Example 2 are significantly greater and aligned in a centripetal fashion around the center of the defect site and the centripetal center, compared to Comparative Example 2. On the contrary, the shape of the bones was non-uniform and the bones were irregularly observed outside the scaffold in Comparative Example 2.

2-2) Histological Assessment

Histological assessment was performed in the same manner as in 1-2) above, except the samples harvested in (2) above were used.

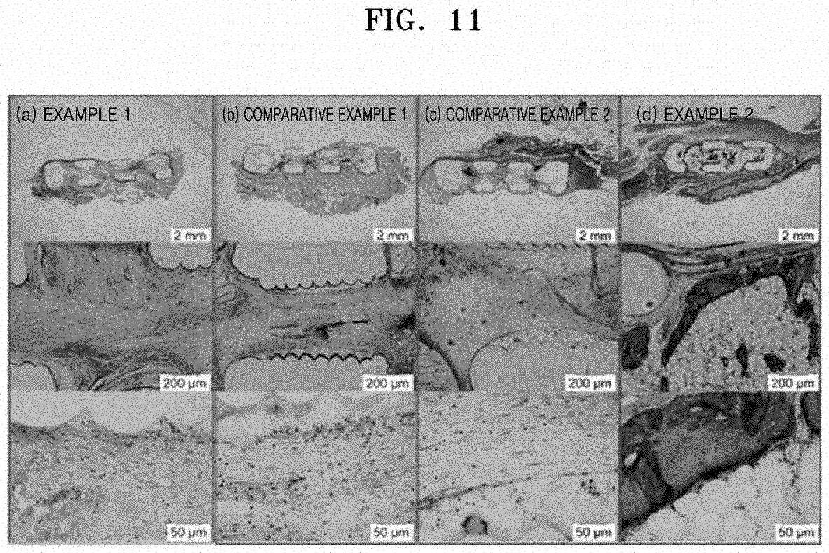

As a result, as shown in FIG. 11, in the case of Example 2 and Comparative Example 2, formation of new bone tissue was confirmed, and particularly, in the case of Example 2, formation of bones inside the scaffold was clearly observed. This is considered that that the biological activity of BMP-2 was maintained and BMP-2 was continuously released over a long period of time in the cage introduced with the biogel even in an environment of bone defects covered with soft tissue and surrounded by fluids. Therefore, the scaffold loaded with the mixed solution of the biogel and BMP-2 may effectively treat bone defects.

(3) Identification of Osteogenesis at Defect Site of Rabbit Tibia

The scaffolds prepared in Examples 1, 2, and 4 and Comparative Example 5 were implanted into detect sites of tibia of rabbits. Specifically, the scaffolds prepared in Examples 1, 2, and 4 and Comparative Example 5 were implanted into the tibia of the rabbits and samples were harvested after 4 weeks. The harvested scaffold samples were scanned with a pixel of 9.85 μm, an Al filter of 0.5 mm, an energy of 59 kV, and a current of 169 μA at a rotation step of 0.4°. Then, the tomographic images were reconstructed using a NRecon package (Bruker, Belgium) and analyzed using a CT Analyzer software (CT-An, Bruker, Belgium). A threshold value of newly formed bone was set based on a native bone. Bone volume (BV) and percent bone volume (BV/TV) of regions of interest (ROIs) that are bones formed inside and outside the scaffold with respect to the defect site, and the results are shown in Table 4 below.

TABLE 4

| Group (no.) | Bone volume ratio (BT/TV) |
|---|---|
| Example 1(8) | 3.85 ± 1.19 |
| Example 2(10) | 6.61 ± 2.82 |
| Example 4(10) | 7.90 ± 2.73 |
| Comparative Example 5(12) | 5.43 ± 1.64 |

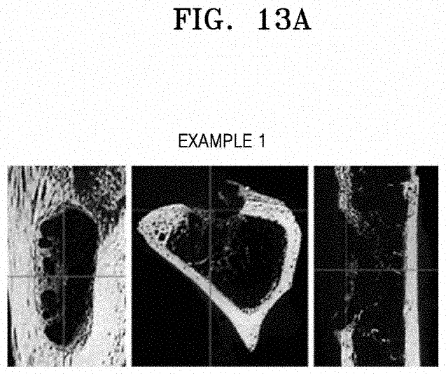

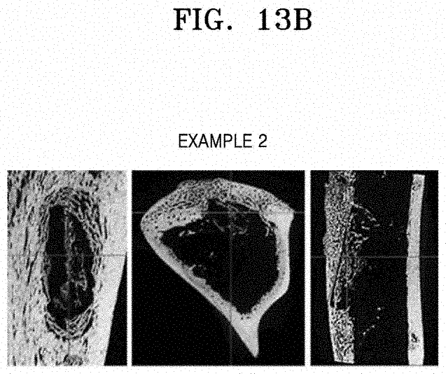

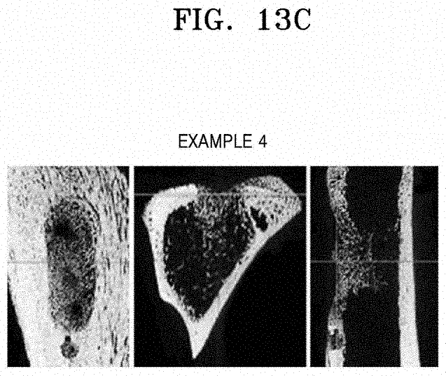

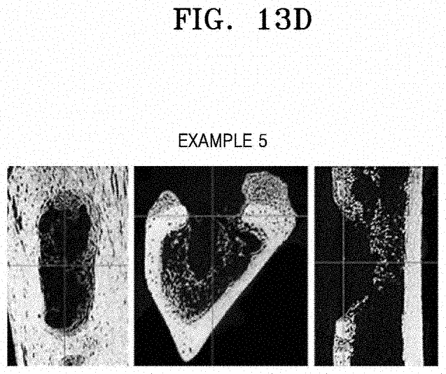

As a result, as shown in Table 4, in the case of Example 4 in which the scaffold is loaded with both BMP-2 and MSCs, the most excellent induction of osteogenesis was observed. Also, in the case of Example 2, it was observed that bones were formed from edges of the defect site and grew inward the scaffold as BMP-2 enclosed in the biogel was gradually released (FIG. 13B). In the case of Comparative Example 5, since MSCs enclosed in the biogel are differentiated to form bone tissue, bones were mainly formed inside the scaffold that is a defect site (FIG. 13D). Also, in the case of Example 4, it was confirmed that bones were formed inside and outside of the scaffold due to maximized osteogenesis by BMP-2 and MSCs. Therefore, excellent therapeutic effects of the mixed solution of the biogel, BMP-2, and MSCs on bone defects were confirmed.

The above description of the disclosure is provided for the purpose of illustration, and it would be understood by those skilled in the art that various changes and modifications may be made without changing technical conception and essential features of the disclosure. Thus, it is clear that the above-described embodiments of the disclosure are illustrative in all aspects and do not limit the disclosure.

1. A biocompatible structure comprising a hollow cage including, in a surface thereof, one or more open chambers recessed inward and holding a biologically active substance.

2. The biocompatible structure of claim 1, wherein the one or more open chambers are formed in portions of top and bottom surfaces of the hollow cage.

3. The biocompatible structure of claim 1, wherein the chambers are formed in two or more layers.

4. The biocompatible structure of claim 2, wherein the open chambers each have a square shape with a length of one side in a range of 0.001 to 10 mm.

5. A biocompatible structure comprising a hollow cage including, in a surface thereof, an open chamber recessed inward and holding a mixed solution of a hydrogel and a biologically active substance.

6. The biocompatible structure of claim 5, wherein the hollow cage is formed of a polymeric material.

7. The biocompatible structure of claim 5, wherein the hydrogel is a mixture of alginate and gelatin.

8. The biocompatible structure of claim 5, wherein the biologically active substance is an osteogenesis-promoting substance.

9. The biocompatible structure of claim 5, wherein the osteogenesis-promoting substance is selected from a bone morphogenetic protein (BMP), a platelet-derived growth factor (PDGF), a transforming growth factor beta (TGF-beta), a basic fibroblast growth factor (bFGF), an insulin-like growth factor 1 (IGF-1), lactoferrin, and bisphosphonate.

10. The biocompatible structure of claim 5, wherein the hydrogel and the biologically active substance are mixed in a weight ratio (w/w) of 0.5:9.5 to 9.5:0.5.

11. The biocompatible structure of claim 5, wherein a cumulative percentage of release of the biologically active substance is from 20% to 99%.

12. The biocompatible structure of claim 5, wherein cells or tissue are further loaded into the open chamber.

13. The biocompatible structure of claim 5, wherein the biologically active substance has initial release stability.

14. A method of manufacturing a biocompatible structure, the method comprising:
preparing a hollow cage including one or more chambers in top and bottom surfaces; and
loading a mixed solution of a hydrogel and a biologically active substance into the chambers.

15. The method of claim 14, wherein the mixed solution of the hydrogel and the biologically active substance is prepared by warming up the hydrogel and adding the biologically active substance to the hydrogel and mixing.

16. The method of claim 15, wherein the warming up is performed at a temperature of 4° C. to 75° C.
